(19) United States
(12) Patent Application Publication
(10) Pub. No.: US 2011/0207691 A1
(43) Pub. Date: Aug. 25, 2011

(54) COBALAMIN COMPOSITIONS FOR THE TREATMENT OF CANCER

(75) Inventor: Chad Brown, Newport Beach, CA (US)

(73) Assignee: BEBAAS, INC., Newport Beach, CA (US)

(21) Appl. No.: 13/023,414

(22) Filed: Feb. 8, 2011

Related U.S. Application Data
(63) Continuation of application No. 11/627,816, filed on Jan. 26, 2007, now abandoned.
(60) Provisional application No. 60/762,131, filed on Jan. 26, 2006.
(60) Provisional application No. 60/765,131, filed on Feb. 3, 2006.

(51) Int. Cl. A61K 31/706; A61K 31/714; A61P 35/00

(52) U.S. Cl. 514/47; 514/52

(57) ABSTRACT

Compositions are provided that contain a combination of a nitric oxide-cobalamin complex along with at least one cobalamin drug conjugate, together with methods for their use in the treatment of neoplastic disease.

This application claims priority to U.S. Provisional Patent Application Ser. No. 60/762,131 filed Jan. 26, 2006 which is incorporated herein by reference in its entirety.

### BACKGROUND OF THE INVENTION

Cancers are a leading cause of death in animals and humans. The exact cause of cancer is not known, but links between certain activities such as smoking or exposure to carcinogens and the incidence of certain types of cancers and tumors has been shown by a number of researchers.

Many types of chemotherapeutic agents have been shown to be effective against cancers and tumor cells, but not all types of cancers and tumors respond to these agents. Unfortunately, many of these agents also destroy normal cells. The exact mechanism for the action of these chemotherapeutic agents are not always known.

Despite advances in the field of cancer treatment the leading therapies to date are surgery, radiation and chemotherapy. Chemotherapeutic approaches are said to fight cancers that are metastasized or ones that are particularly aggressive. Such cytocidal or cytostatic agents work best on cancers with large growth factors, i.e., ones whose cells are rapidly dividing. To date, hormones, in particular estrogen, progesterone and testosterone, and some antibiotics produced by a variety of microbes, alkylating agents, and anti-metabolites form the bulk of therapies available to oncologists. Ideally cytotoxic agents that have specificity for cancer and tumor cells while not affecting normal cells would be extremely desirable. Unfortunately, none have been found and instead agents that target especially rapidly dividing cells (both tumor and normal) have been used.

Clearly, there is a need for better therapies for treating cancer particularly the development of materials that would target cancer cells due to some unique specificity for them.

One promising area of research is in the field of nitric oxide-cobalamin complexes. Cobalamin can be use as a delivery vehicle system and depending on concentration, delivery method and cell condition, nitric oxide can lead to necrotic and/or apoptotic cell death. It has been demonstrated that nitrosylcobalamin appears to have cytostatic properties showing an anti-proliferative effect on WM9 melanoma cells (U.S. Pat. No. 6,752,986). While such an effect exhibits at least a degree of utility in the fight against cancer, room for significant improvement remains.

It is apparent, therefore, that new and improved compositions and methods for anti-neoplastic treatment are greatly to be desired. Specifically, novel efficacious anti-neoplastic cobalamin compositions and methods for their use are highly desirable.

### SUMMARY OF THE INVENTION

It is therefore an object of the invention to provide compositions comprising combinations of a nitric oxide-cobalamin complex and one or more cobalamin. It is a further object to provide methods to use these composition for treating cancer and neoplastic diseases or disorders. Treatment can be accomplished by administering such compositions to subjects in need thereof or contacting neoplastic cells and tissues with said composition.

In accordance with these objects there has been provided a pharmaceutical composition including an effective amount of a nitric oxide-cobalamin complex, at least one cobalamin drug conjugate, and a pharmaceutically acceptable carrier.

In one embodiment, the cobalamin drug conjugate is selected from the group including methylcobalamin, adenosylcobalamin, cyanocobalamin, and hydroxycobalamin.

In another embodiment, the composition includies at least two cobalamin drug conjugates.

In yet another embodiment, the present invention provides any of the above mentioned compositions wherein the composition is an immediate release or a controlled release formulation.

In still another embodiment, the present invention provides any of the above mentioned compositions, wherein the composition is an oral formulation selected from the group including a tablet, a powder, a granule, a lozenge, a gum, a capsule, a pellet and combinations thereof.

In yet still another embodiment, the present invention provides any of the above mentioned compositions, wherein the composition is a topical formulation selected from the group including a gel, a lotion, a patch, a suppository, an iontophoresis solution and combinations thereof.

In a further embodiment, the present invention provides any of the above mentioned compositions, wherein the composition is a formulation selected from the group including an implantable device, a delivery pump, a wafer, a biodegradable polymer and combinations thereof.

The present invention also features a method for inducing cell death in a neoplastic tissue in a subject including administering to the subject an effective amount of a composition as defined above.

In another embodiment, the present invention provides the above mentioned method, wherein the cell death is necrotic cell death, apoptotic cell death or a combination thereof.

In another embodiment, the present invention features a method for treating or ameliorating a neoplastic disease or disorder in a subject including administering to the subject a pharmaceutical composition including an effective amount of a nitric oxide-cobalamin complex, at least one cobalamin drug conjugate, and a pharmaceutically acceptable carrier.

In another embodiment, the present invention provides the above mentioned method, wherein the neoplastic disease or disorder is selected from the group including breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstone tumor, islet cell tumor, primary brain tumor, acute and chronic lymphocyctic and granulocytic tumors, hairy-cell tumor, adenoma, hyperplasia, medullary carcinoma, pheochromocytoma, mucosal neuromas, intestinal ganglioneuromas hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythemia vera, adenocarcinoma, glioblastoma multiforma, acute myeloid leukemia, acute promyelocytic leukemia, acute lymphoblastic leukemia, chronic myelogenous leukemia, myelodysplastic syndrome, lymphomas, malignant melanomas, epidermoid carcinomas and combinations thereof.

In yet another embodiment, the present invention provides the above mentioned method, wherein the treatment facilitates cell death and the cell death is necrotic cell death, apoptotic cell death or a combination thereof.

In still another embodiment, the present invention provides the above mentioned methods, wherein the subject is human.

In yet still another embodiment, the present invention provides the above mentioned methods, wherein the subject is a dog, cat or other domesticated or wild animal.

In a further embodiment, the present invention provides the above mentioned methods, wherein the nitric oxide-cobalamin complex and the at least one cobalamin drug conjugate are administered substantially contemporaneously.

In a yet further embodiment, the present invention provides the above mentioned methods, wherein the nitric oxide-cobalamin complex and the at least one cobalamin drug conjugate are administered sequentially.

In a still further embodiment, the present invention provides the above mentioned methods, wherein the composition is administered at least once a week.

In a yet still further embodiment, the present invention provides the above mentioned methods, wherein the composition is administered at least once a day.

In another embodiment, the present invention provides the above mentioned methods, wherein the dosage of each of the nitric oxide-cobalamin complex and the at least one cobalamin drug conjugate in the composition respectively, is about 1-200 μg/kg of bodyweight of the subject.

In yet another embodiment, the present invention provides the above mentioned methods, wherein the dosage of each of the nitric oxide-cobalamin complex and the at least one cobalamin drug conjugate in the composition respectively, is about 20-100 μg/kg of bodyweight of the subject.

In still another embodiment, the present invention provides the above mentioned methods, wherein the dosage of each of the nitric oxide-cobalamin complex and the at least one cobalamin drug conjugate in the composition respectively, is about 30-50 μg/kg of bodyweight of the subject.

The present invention also features a method for inducing cell death in a neoplastic cell including contracting the neoplastic cell with an effective amount of a pharmaceutical composition including a nitric oxide-cobalamin complex, at least one cobalamin drug conjugate, and a pharmaceutically acceptable carrier.

In another embodiment, the present invention provides the above mentioned method, wherein the nitric oxide-cobalamin complex and the at least one cobalamin drug conjugate are contacted substantially contemporaneously.

In yet another embodiment, the present invention provides the above mentioned method, wherein the nitric oxide-cobalamin complex and the at least one cobalamin drug conjugate are contacted sequentially.

In still another embodiment, the present invention provides the above mentioned method, wherein the cell death is necrotic cell death, apoptotic cell death or a combination thereof.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A is a graph representing the number of BD-MB231 breast cancer cells surviving treatment with hydroxycobalamin, methylcobalamin or adenosylcobalamin and the aforementioned cobalamin analogs in combination with a nitric oxide-cobalamin complex.

FIG. 1B is a graph representing the number of Calu-6 lung cancer cells surviving treatment with hydroxycobalamin, methylcobalamin or adenosylcobalamin and the aforementioned cobalamin analogs in combination with a nitric oxide-cobalamin complex.

FIG. 1C is a graph representing the number of HT-29 colon cancer cells surviving treatment with hydroxycobalamin, methylcobalamin or adenosylcobalamin and the aforementioned cobalamin analogs in combination with a nitric oxide-cobalamin complex.

### DETAILED DESCRIPTION

Compositions and methods are provided that are useful for treating or ameliorating a neoplastic disease such as cancer and inducing cell death in neoplastic tissue and cells. The compositions contain at least two active components: a nitric oxide-cobalamin complex and at least one cobalamin drug conjugate selected from a group consisting of methylcobalamin, adenosylcobalamin, cyanocobalamin and hydroxycobalamin.

The active ingredients can be mixed, together or separately, and instilled in a pharmaceutically acceptable carrier formulation or matrix. Suitable formulations or matrices include, but are not limited to, controlled release tablets, hard or soft capsules, pressed pills, gel caps, dispersible powders or granules, emulsions, and the like. Methods of preparing suitable formulations or matrices are well known in the art. These formulations or matrices are patient-friendly, and permit self-administration of effective amounts of the active compounds. The invention thereby minimizes inconvenience and discomfort for the patient and alleviates the burden and time demands imposed on medical staff.

The compositions of the invention are useful in a method for the treatment of cancer in certain combinations. The specific weight ratio of the respective ingredients in the compositions may be varied when necessary and will depend upon the effective dose of each ingredient or the effective dose of the combination of all the active ingredients in a formulation. Generally, an effective dose of each will be used.

A combination of active ingredients also can be administered separately in the methods of the invention unless specifically indicated otherwise. In addition, active ingredients may be administered in any order and in any subcombination.

Further, compositions of the present invention may be used in combination with other compositions that are used in the treatment/prevention/suppression or amelioration of cancer or neoplastic tissue and cells. Such other compositions (e.g., other anticancer drugs) may be administered, by a route and in an amount commonly used therefore, contemporaneously or sequentially with a composition of the present invention. When a composition of the present invention is used contemporaneously with one or more other compositions such as but not limited to drugs or herbal supplements, vitamin supplements, etc., a pharmaceutical composition may be used that contains the other compositions in addition to the composition of the present invention. Accordingly, the compositions of the present invention include formulations or matrices that contain one or more other active ingredients, in addition to the compositions of the present invention.

### Methods of Making the Pharmaceutical Compositions

In the preferred embodiment, the active ingredients of the pharmaceutical composition of this invention comprise at least a nitric oxide-cobalamin complex along with at least one cobalamin drug conjugate. The nitric oxide-cobalamin complex may be prepared as described in U.S. Pat. No. 5,936,082 which is herein incorporated by reference in its entirety. The cobalamin drug conjugate comprises at least one cobalamin selected from a group consisting of methylcobalamin, cyanocobalamin, adenosylcobalamin, and hydroxycobalamin. A suitable pharmaceutical carrier may be used if necessary.

The term “composition” or a “formulation” as used herein is intended to encompass a product comprising the specified active ingredients in the specified amounts, as well as any product which results, directly or indirectly, from the combination of the specified active ingredients in the specified amounts. Such term is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the active compounds of the present invention and a pharmaceutically acceptable carrier.

Generally, the terms “active ingredients” or “compounds” of the invention describe types of cobalamins or cobalamin complexes including, but not limited to nitric oxide-cobalamin complex, methylcobalamin, adenosylcobalamin, cyanocobalamin, and hydroxycobalamin. The active ingredients are used in different mixtures containing varying effective amounts of each active compound of the invention, which would be suitable for the treatment of different types of cancer.

### Dosage Formulations

The pharmaceutical compositions of this invention conveniently are presented in dosage unit forms and may be prepared by methods that are well known in the art of pharmacy. Suitable methods are described in, for example, Remington, The Science and Practice of Pharmacy, ed. Gennaro et al., 20th Ed. (2000), although the skilled artisan will recognize that other methods are known and are suitable for preparing the compositions. All methods include the step of bringing the active ingredients into association with the carrier which constitutes one or more accessory ingredients. In general, the pharmaceutical compositions are prepared by uniformly and intimately bringing the active ingredients into association with a liquid carrier or a finely divided solid carrier or both, and then, if necessary, shaping the product into the desired formulation. In the pharmaceutical composition the active ingredients are included in an effective amount sufficient to produce the desired effect upon the process or condition of diseases.

The pharmaceutical compositions containing the active ingredients can be in a form suitable for oral use, for example, as tablets. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredients in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Inert ingredients are components such as pharmaceutically acceptable carriers, adjuvant, diluents or excipients, etc., that are compatible with the active ingredients of the formulation and that are not deleterious to the recipient thereof.

Formulations suitable for oral administration can also be presented as hard gelatin capsules wherein the active ingredients are mixed with an inert solid diluent, for example, calcium carbonate, calcium phosphate or kaolin; or as soft gelatin capsules wherein the active ingredients are mixed with water or an oil medium, for example peanut oil, liquid paraffin, or olive oil.

Liquid formulations can include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

Other formulations include but are not limited to powders, granules, lozenges, gum, pellets and combinations thereof. Topical formulations such as gels, lotions, patches, iontophoresis solutions or combinations thereof may also be employed.

A composition according to the present invention can be instilled in a carrier matrix, such as controlled release pills, tablets, hard or soft capsules, pressed pills, gel caps, dispersible powders or granules, etc., all for patient-friendly, self-administration of effective amounts of the composition. A patient-friendly pharmaceutical composition can be helpful for treating for example, human subjects. In this regard, the skilled artisan will appreciate subjects to include at least dogs, cats and other such domesticated or wild animals.

Further, one feature of the present invention is a mixture of compositions. A “mixture” is a combination containing different types of active ingredients defined above, each in effective amounts, useful for the treatment of cancer or against neoplastic tissue and/or cells.

### Methods of Treatment

It is an object of the present invention to provide patient-friendly modes of delivery to patients of such effective amounts of the active ingredients. For this purpose, oral administration is a preferable mode as it reduces the inconvenience and discomfort of subcutaneous and intramuscular injections. Other features of administration of an effective amount of active ingredients such can be employed as and when necessary and include at least implantable device, delivery pump, wafers, biodegradable polymers or combinations thereof may be employed as and when necessary.

The compositions of the present invention can be administered in an effective amount. An “effective amount” is meant that amount, which when administered, either alone or in combination, is sufficient to effect the treatment of cancer or neoplastic tissue and cells. In general, an effective amount is any amount that can cause the death of a neoplastic cell or tissue, or can treat or ameliorate a neoplastic disease or disorder of a patient. Such amounts will depend on at least the particular neoplasm and in the case of treating a subject, the severity of the condition and individual patient parameters.

At the outset it must be noted that the terms “administration of” and/or “administering a” compound should be understood to mean at least providing the active compounds of the invention, in any formulation, to an individual in need of treatment.

### Administration of a Composition

The composition according to the present invention can be administered in oral preparations such as tablets, powders, granules, lozenges, gum, capsules, pellets or combinations thereof. In a preferred embodiment, a tablet, or a hard or soft capsule can be preferably absorbed directly via the mucosa, such as buccal or nasal mucosa, into the blood stream before being subjected to digestion and degradation in the liver. A preferred formulation can include fast absorbing capsules or tablets, etc. Other preferred embodiments can include time release or delayed release formulations for slower or maintained absorption.

In a preferred construction, the composition according to the present invention can be administered in a patient-friendly effective amount as a topical or a transdermal formulation. A topical or transdermal formulation allows for ease of application and includes at least gels, lotions, patches, iontophoresis solutions, or combinations thereof. Additionally, the composition according to the present invention can be administered in a patient-friendly effective amount via an implantable device, a delivery pump, a wafer, a biodegradable polymer, or combinations thereof.

Also in accordance with the present invention, compositions can be administered by injection, that is, intravenously, intramuscularly, intracutaneously, subcutaneously, intraduodenally, or intraperitoneally. Also, the compositions of the present invention can be administered by inhalation, for example, intranasally.

The skilled artisan will appreciate that release rate of the composition according to the present invention can be varied. The composition can be administered as an immediate release composition or as a controlled or delayed release formulation. Controlled release formulations can result in a more uniform composition release over time possibly alleviating aspects of some side effects of treatment. Conversely, immediate release formulations can be useful for rapidly increasing a concentration at a target site.

A preferable dosage range comprises administering about 1-200 microgram of each active ingredient per kilogram of bodyweight of a subject. A more preferable dosage range would be about 20-100 microgram of each active ingredient per kilogram of bodyweight of a subject. A dosage range of about 30-50 microgram of each active ingredient per kilogram of bodyweight of a subject would be further preferred.

Thus, the formulations of the present invention may be administered preferably, but are not limited to, oral routes of administration and may be formulated, alone or together, in suitable dosage unit formulations containing conventional non-toxic pharmaceutically acceptable carriers, adjuvants and vehicles appropriate for each route of administration.

### Dosing

The skilled artisan will appreciate that the combination of active ingredients can be administered separately in the methods of the invention unless specifically indicated otherwise. In addition, active ingredients may be administered in any order and in any subcombination. Any order will be appreciated in the art to include at least contemporaneous and sequential administration. Contemporaneous indicating administration at substantially the same time and sequential indicating a first administration followed by at least a second administration.

It should be appreciated that administration of a composition according to the present invention can include a dosing time course. A preferable dosing time course can include administration of a composition according to the present invention at least once a week. Another preferable dosing time course can include administration of a composition according to the present invention at least once a day. The skilled artisan will further appreciate that the dosing time course can be varied, modified or altered according to factors such as, but not limited to, the dosage range, the composition administered, or the progression of the neoplastic disease or disorder.

### Cell Death

The present invention can include induction of neoplastic cell death by administering an effective amount of a composition according to the present invention. Administration of an effective amount of a composition can lead to necrotic cell death, apoptotic cell death or to a combination of necrosis and apoptosis. Research has demonstrated that varying the amount available nitric oxide, the exposure time and the cell type exposed alters the manner of cell death. In addition to nitric oxide conditions, the cellular milieu also appears to effect cell death type. For example, nitric oxide can inhibit cytochrome oxidase in cells exposed to low amounts of oxygen wherein gylcolysis is insufficient to compensate thus leading to necrotic dell death (Borutaite and Brown, Biochemical Society Transactions (2005) volume 33, part 6, pages 1394-1396). Further, nitric oxide can also induce apoptotic cell death via the extrinsic apoptotic pathway. Nitric oxide can bind to a tumor necrosis factor receptor, part of the death receptor super family, and initiate the programmed cell death cascade such as has been shown in NIH-OVCAR-3 cells (Bauer et al. Journal of the National Cancer Institute, vol. 94, No. 13, pages 1010-1019, 2002).

Cell death, necrotic, apoptotic or a combinations thereof, can occur in a subject, e.g., a human, suffering from a neoplastic disease or disorder after treatment with a composition of the present invention. As the skilled artisan will appreciate, the neoplastic disease or disorder can be a sarcoma or a carcinoma.

While not wishing to be bound by theory, it appears that the combination of a nitric oxide-cobalamin complex and a cobalamin drug conjugate increases the efficacy of the anti-neoplastic effects, i.e., apoptosis and/or necrosis, of nitric oxide.

### Kits

Since the present invention has an aspect that relates to the treatment or amelioration of the disease or disorder described herein with a combination of active agents which may be administered together or separately, the invention also relates to combining active agents in kit form. The kit can include one or more containers having the nitric oxide-cobalamin complex packaged separately or together with a cobalamin drug conjugate. These active agents can be presented in unit dosage form or as powders, liquids or other material suitable for reconstitution to a desired dosage.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

### EXAMPLES

The present invention is further described in the following examples, which do not limit the scope of the invention described in the claims.

Cell cultures were grown and maintained using standard protocols for the respective cell line. Suitable culturing methods are described in, for example, Cancer Cell Culture: Method and Protocols (Methods in Molecular Medicine) Langdon (Ed) Humana Press, 2003 and General Techniques of Cell Culture (Handbooks in Practical Animal Cell Biology) Harrison and Rae, Cambridge University Press, 2005. Cultures were grown for 48 hours and subsequently counted to determine the number surviving after treatment with hydroxycobalamin, methylcobalamin or adenosylcobalamin and the aforementioned cobalamin analogs in combination with a nitric oxide-cobalamin complex. Determination of cell survival can be accomplished using, for example, a hemocytometer and Trypan Blue staining. In this technique, cells are counted after staining with the Trypan Blue because dead cells stain, whereas living cells do not. Total number of cells can be determined living and dead cells can be determined and treatments compared. Results are listed below.

### Example 1

### BD-MB231 Breast Cancer Cells

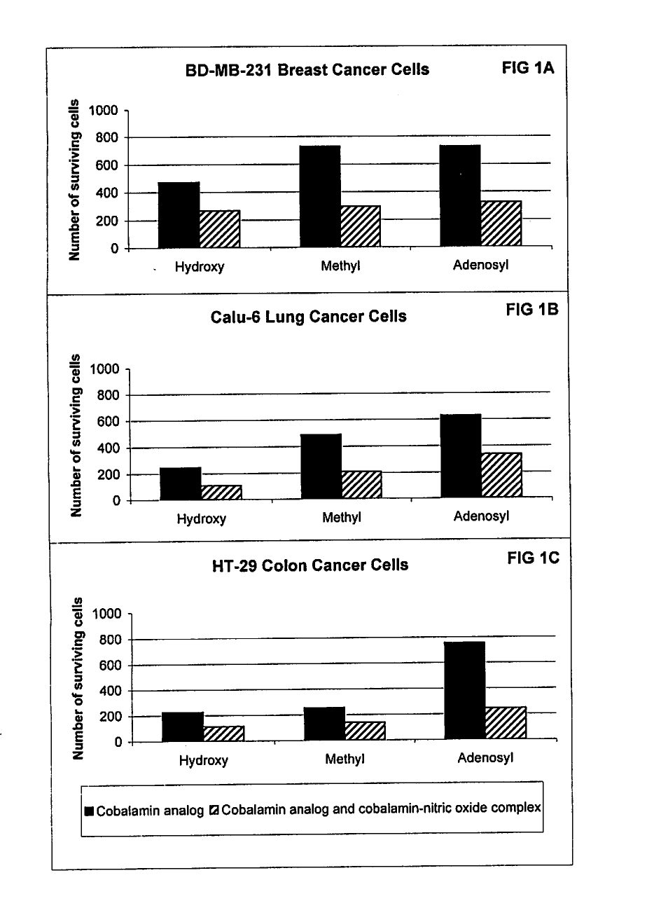

FIG. 1A shows the number of surviving BD-MB231 breast cancer cells after treatment with hydroxycobalamin, methylcobalamin or adenosylcobalamin and the aforementioned cobalamin analogs in combination with a nitric oxide-cobalamin complex. As can be seen in the figure, the combination of cobalamin analog and nitric oxide-cobalamin complex resulted a statistically significant decrease in the number of surviving cancer cells after treatment. Data showing the decrease in survival can be found in Table 1A wherein 1) administration of hydroxycobalamin combined with a nitric oxide-cobalamin complex resulted in a decrease in the number of surviving cells from a mean of 472 to a mean of 269 when compared to administration of hydroxycobalamin alone; 2) administration of methylcobalamin combined with a nitric oxide-cobalamin complex resulted in a decrease in the number of surviving cells from a mean of 729 to a mean of 296 when compared to administration of hydroxycobalamin alone; and 3) administration of adenosylcobalamin combined with a nitric oxide-cobalamin complex resulted in a decrease in the number of surviving cells from a mean of 728 to a mean of 324 when compared to administration of hydroxycobalamin alone.

### Example 2

### Calu-6 Lung Cancer Cells

FIG. 18 shows the number of surviving Calu-6 lung cancer cells after treatment with hydroxycobalamin, methylcobalamin or adenosylcobalamin and the aforementioned cobalamin analogs in combination with a nitric oxide-cobalamin complex. As can be seen in the figure, the combination of cobalamin analog and nitric oxide-cobalamin complex resulted a statistically significant decrease in the number of surviving cancer cells after treatment. Data showing the decrease in survival can be found in Table 1B wherein 1) administration of hydroxycobalamin combined with a nitric oxide-cobalamin complex resulted in a decrease in the number of surviving cells from a mean of 247 to a mean of 108 when compared to administration of hydroxycobalamin alone; 2) administration of methylcobalamin combined with a nitric oxide-cobalamin complex resulted in a decrease in the number of surviving cells from a mean of 489 to a mean of 210 when compared to administration of hydroxycobalamin alone; and 3) administration of adenosylcobalamin combined with a nitric oxide-cobalamin complex resulted in a decrease in the number of surviving cells from a mean of 632 to a mean of 337 when compared to administration of hydroxycobalamin alone.

### Example 3

### HT-29 Colon Cancer Cells

FIG. 1C shows the number of surviving HT-29 colon cancer cells after treatment with hydroxycobalamin, methylcobalamin or adenosylcobalamin and the aforementioned cobalamin analogs in combination with a nitric oxide-cobalamin complex. As can be seen in the figure, the combination of cobalamin analog and nitric oxide-cobalamin complex resulted a statistically significant decrease in the number of surviving cancer cells after treatment. Data showing the decrease in survival can be found in Table 1C wherein 1) administration of hydroxycobalamin combined with a nitric oxide cobalamin complex resulted in the decrease in number of surviving cells from a mean of 227 to a mean of 115 when compared to administration of hydroxycobalamin alone; 2) administration of methylcobalamin combined with a nitric oxide-cobalamin complex resulted in a decrease in the number of surviving cells from a mean of 256 to a mean of 141 when compared to administration of hydroxycobalamin alone; and 3) administration of adenosylcobalamin combined with a nitric oxide-cobalamin complex resulted in a decrease in the number of surviving cells from a mean of 755 to a mean of 247 when compared to administration of hydroxycobalamin alone.

Further modifications and alternative embodiments of this invention will be apparent to those skilled in the art in view of the description. Accordingly, this description is to be construed as illustrative only and is for the purpose of teaching those skilled in the art the manner of carrying out the invention. It is to be understood that the forms of the invention herewith shown and described are to be taken as the presently preferred embodiments, Various changes may be made for example, equivalent elements or materials may be substituted for those illustrated and described herein and certain features of the invention may be utilized independently of the use of other features, all as would be apparent to one skilled in the art after having the benefit of this description of the invention.

What is claimed:

1. A pharmaceutical composition comprising an effective amount of a nitric oxide-cobalamin complex, at least one cobalamin drug conjugate, and a pharmaceutically acceptable carrier.

2. The composition according to claim 1, wherein said cobalamin drug conjugate is selected from the group consisting of methylcobalamin, adenosylcobalamin, cyanocobalamin, hydroxycobalamin.

3. The composition according to claim 2, comprising at least two cobalamin drug conjugates.

4. The composition according to claim 1, wherein said composition is an immediate release or a controlled release formulation.

5. The composition according to claim 1, wherein said composition is an oral formulation selected from the group consisting of a tablet, a powder, a granule, a lozenge, a gum, a capsule, a pellet and combinations thereof.

6. The composition according to claim 1, wherein said composition is a topical formulation selected from the group consisting of a gel, a lotion, a patch, a suppository, an iontophoresis solution and combinations thereof.

7. The composition according to claim 1, wherein said composition is a formulation selected from the group consisting of an implantable device, a delivery pump, a wafer, a biodegradable polymer and combinations thereof.

8. A method for inducing cell death in a neoplastic tissue in a subject comprising administering to said subject an effective amount of a composition according to claim 1.

9. A method for treating or ameliorating a neoplastic disease or disorder in a subject comprising administering to said subject a pharmaceutical composition comprising an effective amount of a nitric oxide-cobalamin complex, at least one cobalamin drug conjugate, and a pharmaceutically acceptable carrier.

10. The method according to claim 9, wherein said neoplastic disease or disorder is selected from the group consisting of breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstone tumor, islet cell tumor, primary brain tumor, acute and chronic lymphocyctic and granulocytic tumors, hairy-cell tumor, adenoma, hyperplasia, medullary carcinoma, pheochromocytoma, mucosal neuromas, intestinal ganglioneuromas hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic sarcoma, malignant hypercalcemia, renal cell tumor, polycythemia vera, adenocarcinoma, glioblastoma multiforma, acute myeloid leukemia, acute promyelocytic leukemia, acute lymphoblastic leukemia, chronic myelogenous leukemia, myelodysplastic syndrome, lymphomas, malignant melanomas, epidermoid carcinomas and combinations thereof.

11. The method according to claim 9, wherein said treatment facilitates cell death and said cell death is necrotic cell death, apoptotic cell death or a combination thereof.

12. The method according to claim 9, wherein said subject is human.

13. The method according to claim 9, wherein said subject is a clog, cat or other domesticated or wild animal.

14. The method according to claim 9, wherein said nitric oxide-cobalamin complex and said at least one cobalamin drug conjugate are administered substantially contemporaneously.

15. The method according to claim 9, wherein said nitric oxide-cobalamin complex and said at least one cobalamin drug conjugate are administered sequentially.

16. The method according to claim 9, wherein said composition is administered at least once a week.

17. The method according to claim 9, wherein said composition is administered at least once a day.

18. The method according to claim 9, wherein the dosage of each of said nitric oxide-cobalamin complex and said at least one cobalamin drug conjugate in said composition respectively, is about 1-200 μg/kg of bodyweight of said subject.

19. The method according to claim 9, wherein the dosage of each of said nitric oxide-cobalamin complex and said at least one cobalamin drug conjugate in said composition respectively, is about 20-100 μg/kg of bodyweight of said subject.

20. The method according to claim 9, wherein the dosage of each of said nitric oxide-cobalamin complex and said at least one cobalamin drug conjugate in said composition respectively, is about 30-50 μg/kg of bodyweight of said subject.

21. A method for inducing cell death in a neoplastic cell comprising contracting said neoplastic cell with an effective amount of a pharmaceutical composition comprising a nitric oxide-cobalamin complex, at least one cobalamin drug conjugate, and a pharmaceutically acceptable carrier.

22. The method according to claim 21, wherein said nitric oxide-cobalamin complex and said at least one cobalamin drug conjugate are contacted substantially contemporaneously.

23. The method according to claim 21, wherein said nitric oxide-cobalamin complex and said at least one cobalamin drug conjugate are contacted sequentially.

24. The method according to claim 21, wherein said cell death is necrotic cell death, apoptotic cell death or a combination thereof.
